(19) United States
(12) Patent Application Publication
(10) Pub. No.: US 2026/0216387 A1
(43) Pub. Date: Jul. 30, 2026

(54) ROOM IRRADIATION CONFIGURATION WITH ULTRAVIOLET RADIATION CONFIGURATION

(71) Applicant: AMICO MOBILITY SOLUTIONS CORPORATION, Richmond Hill (CA)

(72) Inventors: Wayne BENSON, Richmond Hill (CA); Habib-Ur Rehman ABBASI, Richmond Hill (CA)

(21) Appl. No.: 19/461,023

(22) Filed: Jan. 27, 2026

Related U.S. Application Data
(60) Provisional application No. 63/750,531, filed on Jan. 28, 2025.

(51) Int. Cl. A61L 2/08; A61G 7/10; A61L 2/26; A61L 103/15; A61L 103/75

(52) U.S. Cl. CPC A61L 2/08; A61G 7/1073; A61L 2/26; A61L 2103/15; A61L 2103/75; A61L 2202/11

(57) ABSTRACT

Disclosed herein is a room irradiation configuration for exposing a room to ultraviolet (UV) radiation, comprising a patient lift displacement configuration, comprising: a mount; and a patient lift coupler, operably coupled to the mount such that it is displaceable, relative to the mount, and is configured to operably couple to a patient lift, the patient lift coupler comprising a UV radiation configuration for emitting UV radiation; and a controller operably coupled to the UV radiation configuration to adjust a characteristic of the emitted UV radiation. While the mount is mounted to the room, and the UV radiation configuration is emitting UV radiation: a first portion of the room is exposed to the UV radiation; and the patient lift coupler is displaceable, relative to the room, via the displaceability of the patient lift coupler, relative to the mount, for exposing another portion of the room to the UV radiation.

### CROSS-REFERENCE TO RELATED APPLICATIONS

This application claims benefit of U.S. Provisional Patent Application Ser. No. 63/750,531, filed Jan. 28, 2025, which is incorporated herein by reference in its entirety.

### FIELD

The present application relates to a room irradiation configuration, and in particular, to a room irradiation configuration configured to expose a room to ultraviolet radiation.

### BACKGROUND

A patient lift is configured to assist in the displacement of a patient who may have limited mobility. Existing patient lifts include a strap that is connectible to a patient supporter such as a sling or a seat, upon which the patient may be supported. While the patient is supported on the patient supporter, the strap is extendible or retractable to displace the patient. Existing patient lifts are displaceable relative to a room in which the patient lift is disposed. It is desirable to disinfect the room in which the patient lift is disposed for enhancing safety and hygiene and for promoting infection control standards, particularly in healthcare and medical environments.

### SUMMARY

In one aspect, there is provided a room irradiation configuration configured for exposing a room to ultraviolet (UV) radiation, comprising: a patient lift displacement configuration, comprising: a mount configured to be mounted to the room; and a patient lift coupler, operably coupled to the mount such that the patient lift coupler is displaceable, relative to the mount, and is configured to operably couple to a patient lift such that the patient lift is displaceable, relative to the patient lift coupler, the patient lift coupler comprising a UV radiation configuration configured to emit UV radiation; a controller operably coupled to the UV radiation configuration to adjust a characteristic of the UV radiation emitted by the UV radiation configuration; wherein, while the mount is mounted to the room, and the UV radiation configuration is emitting UV radiation: a first portion of the room is exposed to the UV radiation; and the patient lift coupler is displaceable, relative to the room, via the displaceability of the patient lift coupler, relative to the mount, for exposing another portion of the room to the UV radiation.

Other aspects will be apparent from the description and drawings provided herein.

### BRIEF DESCRIPTION OF DRAWINGS

In the figures, which illustrate example embodiments,

FIG. 1 is a schematic diagram of an example embodiment of a room irradiation configuration;

FIG. 2 is a schematic diagram depicting the software structure of the room irradiation configuration of FIG. 1;

FIG. 3 is a schematic diagram of an example embodiment of a patient lift operably couplable to the room irradiation configuration of FIG. 1; and

FIG. 4 is a schematic diagram of another example embodiment of the room irradiation configuration of FIG. 1.

### DETAILED DESCRIPTION

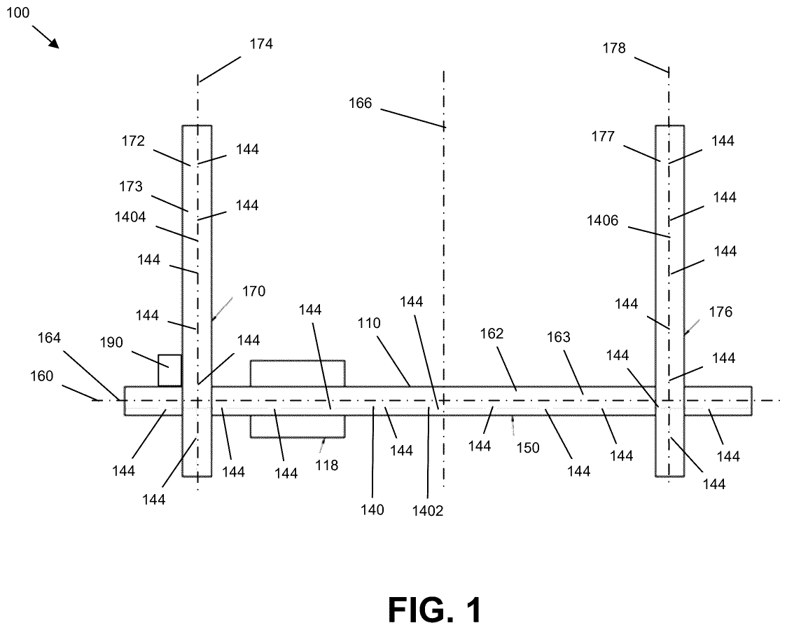

Disclosed herein in a room irradiation configuration 100, as depicted in FIG. 1, that is configured to emit ultraviolet (UV) radiation from a UV radiation configuration 140 of a patient lift displacement configuration 110. In some embodiments, for example, the patient lift displacement configuration 110 includes a mount 170 configured to be mounted to the room, and further includes a patient lift coupler 150 that is operably coupled to the mount 170 such that the patient lift coupler 150 is displaceable, relative to the mount 170. The patient lift coupler 150 comprises the UV radiation configuration 140 that is configured to emit UV radiation. The characteristic of the UV radiation emitted by the UV radiation configuration 140 (e.g. wavelength and intensity of the emitted UV radiation) can be adjusted. While the room irradiation configuration 100 is disposed in a room, for example, while the room irradiation configuration 100 is mounted to the room via mounting of the mount 170 to the room (e.g. while the mount 170 is mounted to the room), and the UV radiation configuration 140 is emitting UV radiation, a portion of the room is exposed to the UV radiation emitted by the UV radiation configuration 140. The patient lift coupler 150 is displaceable, relative to the room, via the displaceability of the patient lift coupler 150, relative to the mount 170, for exposing another portion of the room to the UV radiation. The patient lift coupler 150 is configured to operably couple to a patient lift 118 such that the patient lift 118 is displaceable, relative to the patient lift coupler 150.

In some embodiments, for example, there is a need for effective disinfection solutions, for example, to effectuate microbial reduction and reduction of pathogen viability. Such a need is particularly present in healthcare settings, where the risk of infection transmission is high. UV disinfection has emerged as a method for eliminating pathogens, including bacteria, viruses, and fungi, from various surfaces. In some embodiments, for example, by exposing pathogens and microorganisms to UV radiation, the DNA and RNA of the pathogens and microorganisms are disrupted. In some embodiments, for example, such disruption of the DNA and RNA of the pathogens and microorganisms renders the pathogens and microorganisms inactive and unable to reproduce, which, in some embodiments, for example, is effective for inactivating a broad spectrum of microorganisms and pathogens. In some embodiments, for example, the inactivation of microorganisms and pathogens is with effect that the surface, on which the pathogens and microorganisms are disposed, is disinfected and sanitized, at least with respect to the pathogens and microorganisms.

In some embodiments, for example, by exposing the portion of the room, in which the room irradiation configuration 100 is disposed, to the UV radiation emitted by the UV radiation configuration 140, the DNA and RNA of the pathogens and microorganisms disposed in the portion of the room are disrupted, with effect that the pathogens and microorganisms become inactive and are unable to reproduce, which, in some embodiments, for example, effectuates disinfecting and sanitizing of the portion of the room that is exposed to the UV radiation.

By adjusting the UV radiation wavelength and intensity of the UV radiation emitted by the UV radiation configuration 140 to the portion of the room and the duration of exposure of UV radiation to the portion of the room, the dosage of UV radiation emitted to the portion of the room can be controlled, and therefore, the disinfection and sanitation of the room can be controlled. In some embodiments, for example, the room irradiation configuration 100 is configured to deliver the optimal dosage of UV radiation required to achieve effective disinfection of the room without damaging the patient lift displacement configuration 110 or the room, or posing risks to users.

In some embodiments, for example, by disinfecting and sanitizing the room, a safer and more hygienic environment is provided to users, such as patients and healthcare providers.

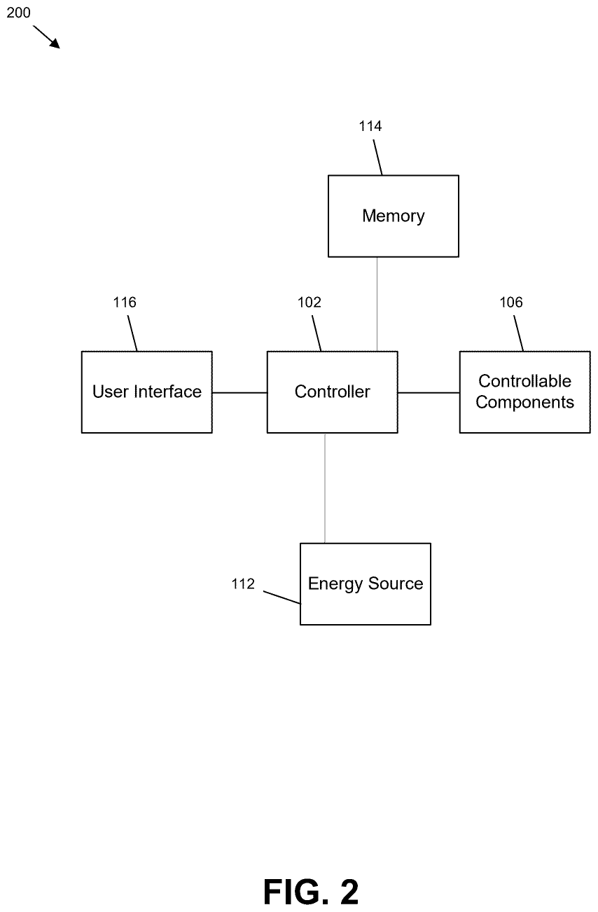

FIG. 2 is a schematic diagram showing the software structure 200 of the room irradiation configuration 100 in some embodiments. In some embodiments, for example, the room irradiation configuration 100 comprises a control module or a controller 102 disposed in operable communication with controllable components 106, a user interface 116, an energy source 112, and a memory 114, as described in greater detail below. In some embodiments, for example, the controller 102 is the controller of the patient lift 118 that is operably coupled to the patient lift coupler 150.

In some embodiments, for example the room irradiation configuration 100 comprises a plurality of components including, e.g., a controller 102, memory 114 (volatile and/or non-volatile memory, e.g., RAM, ROM, EEPROM, and/or the like), non-removable or removable memory (e.g., hard disk drives, CD-ROMs, DVDs, solid-state memory, flash memory, and/or the like), networking components for connecting to a network, a plurality of controllable components 106 including a UV radiation configuration 140, and a system bus coupling the various components to the controller 102.

In some embodiments, for example the room irradiation configuration 100 comprises a user interface 116 to effectuate interaction between the room irradiation configuration 100 and a user. In some embodiments, for example, the user interface 116 includes a display such as a display screen or a touchscreen, one or more input devices such as buttons, a hand control unit, a control panel that is, for example, mounted to the room, a keyboard and a computer mouse, other input/output devices such as a microphone, a speaker, a printer, a scanner, and the like. In some embodiments, for example, the user interface 116 is the user interface of the patient lift 118 that is operably coupled to the patient lift coupler 150.

In some embodiments, the controller 102 includes a processor or a central processing unit (CPU) that is operably coupled to a memory 114 such as a ROM, RAM, persistent memory, or flash memory for storing data, and input or output peripherals. In some embodiments, for example, the controller 102 functions as a central controller for controlling all of the communications of the room irradiation configuration 100. In some embodiments, for example, the controller 102 further functions as a central controller for controlling the communications between the room irradiation configuration 100 and an external server or user equipment, such as a computer, laptop, smart device, a control panel in a control room, and the like.

In some embodiments, for example, the processor is one or more single-core or multiple-core computing processors such as INTEL® microprocessors (INTEL is a registered trademark of Intel Corp., Santa Clara, CA, USA), AMD® microprocessors (AMD is a registered trademark of Advanced Micro Devices Inc., Sunnyvale, CA, USA), ARM® microprocessors (ARM is a registered trademark of Arm Ltd., Cambridge, UK) manufactured by a variety of manufactures such as Qualcomm of San Diego, California, USA, under the ARM® architecture, or the like.

In some embodiments, for example, the processor is one or more real-time processors, programmable logic controllers (PLCs), microcontroller units (MCUs), μ-controllers (UCs), specialized or customized processors or controllers using e.g., field-programmable gate array (FPGA) or application-specific integrated circuit (ASIC) technologies, and/or the like.

The controller 102 communicates with the controllable components 106, the user interface 116, the energy source 112, and the memory 114. In some embodiments, the controller 102 receives data or instructions, saves them to a memory, and processes the received data or instructions. The data or instructions may be real time or historical data or instructions. In some embodiments, the controller 102 processes the data or instructions by, for example, comparing them with one or more preset thresholds.

In some embodiments, the controller 102 is configured to control the functioning or operation of the room irradiation configuration 100. In some embodiments, for example, based on the data from a component of the room irradiation configuration 100, such as a controllable component 106 or the energy source 112, the controller 102 sends a control command to the user interface 116 to render a graphic representative of the data, or sends a control command to one or more of the controllable components 106 to operate the room irradiation configuration 100.

In some embodiments, the room irradiation configuration 100 includes a user interface 116 operably coupled to the controller 102 to receive instructions from a user, and to communicate information to a user. In some embodiments, for example, the user interface 116 includes an input device, such as user equipment, a keyboard, mouse, camera, touch screen, one or more buttons, a hand control unit, a microphone, a control panel, and the like. In some embodiments, for example, the user interface 116 incudes an output device, such as a display screen or touch screen, a speaker, a printer, a scanner, the hand control unit, and the like. In some embodiments, for example, the user interface 116 is configured to operably couple the controller 102 to an external input device, such as a user device (e.g. computer, smartphone, etc.), for the controller 102 to receive instructions from the external input device. In some embodiments, for example, the user interface 116 is configured to operably couple the controller 102 to an external output device to communicate information to the external output device. In some embodiments, the controller 102 is configured to send a control command to the user interface 116 for displaying a graphical representation of data that is received by the controller 102. In some embodiments, the user interface 116, via input from a user, is configured to send a control command to the controller 102 for controlling the room irradiation configuration 100. In some embodiments, for example, a user can input a control command to the controller 102 via the user interface 116 to adjust the characteristics of the UV radiation emitted from the UV radiation configuration 140, select the environment in which the room irradiation configuration 100 is disposed, select the operating mode of the room irradiation configuration 100, adjust the displacement of the patient lift coupler 150, relative to the mount 170, and the like.

In some embodiments, for example, the controller 102 sends a control command to the user interface 116 to generate a graphical representation of the operating status of the room irradiation configuration 100. In some embodiments, for example, the controller 102 sends a control command to the user interface 116 to generate a graphical representation of a menu of options, and the user can input a control command using the user interface 116 to select an option from the menu, and the user interface 116 sends the control command to the controller 102 to control the operation of the room irradiation configuration 100. In some embodiments, for example, the menu of options includes a list of possible environments in which the room irradiation configuration 100 is disposed. In some embodiments, for example, the menu of options includes a list of operating modes of the room irradiation configuration 100.

The controller 102 receives instructions from a user via the user interface 116 or data from one or more components of the room irradiation configuration 100 to make a plurality of decisions, such as characteristic of the UV radiation to be emitted by the UV radiation configuration 140, the environment of the room irradiation configuration 100, the operating mode of the room irradiation configuration 100, the displacement of the patient lift coupler 150, relative to the mount 170, and the like, based on the received user instructions or data. The controller 102 then instructs the controllable components 106 to function accordingly.

In some embodiments, for example, the room irradiation configuration 100 includes an energy source 112 for providing energy to the components of the room irradiation configuration 100, such as the controller 102, memory 114, user interface 116, and controllable components 106. In some embodiments, for example, the energy source 112 is the energy source of the patient lift 118. In some embodiments, for example, the energy source is an electrical energy source, such as a battery. In some embodiments, for example, the energy source is a fuel cell. In some embodiments, the room irradiation configuration 100 is operably coupled with an external energy source, such as a portable battery, portable generator, external battery, and the like. In some embodiments, for example, the room irradiation configuration 100 is wirelessly connectable to the external energy source for wirelessly energizing the room irradiation configuration 100.

In some embodiments, for example, the controller 102 is operably coupled to the energy source 112 for adjusting the amount of energy provided to the controllable components 106.

FIG. 1 depicts an example embodiment of the room irradiation configuration 100. In some embodiments, for example, the room irradiation configuration 100 includes a patient lift displacement configuration 110.

In some embodiments, for example, the patient lift displacement configuration 110 includes a mount 170 configured to be mounted to the room. In some embodiments, for example, the mount 170 is mountable to the room via mechanical fasteners (e.g. nuts and bolts, screws, and the like). In some embodiments, for example, the patient lift displacement configuration 110 further includes a patient lift coupler 150 that is operably coupled to the mount 170 such that the patient lift coupler 150 is displaceable, relative to the mount 170. The patient lift coupler 150 comprises a UV radiation configuration 140 configured to emit UV radiation. The patient lift coupler 150 is configured to operably couple to a patient lift 118 such that the patient lift 118 is displaceable, relative to the patient lift coupler 150.

In some embodiments, for example, the room irradiation configuration 100 further includes the energy source 112. In some embodiments, for example, the energy source 112 is operably coupled to the UV radiation configuration 140 to provide energy to the UV radiation configuration 140.

In some embodiments, for example, the room irradiation configuration 100 further includes the controller 102. The controller 102 is operably coupled to the UV radiation configuration 140 to adjust a characteristic of the UV radiation emitted by the UV radiation configuration 140.

While the room irradiation configuration 100 is disposed in a room, for example, while the room irradiation configuration 100 is mounted to the room via mounting of the mount 170 to the room (e.g. while the mount 170 is mounted to the room), and the UV radiation configuration 140 is emitting UV radiation, a portion of the room (e.g. a first portion of the room) is exposed to the UV radiation emitted by the UV radiation configuration 140. The patient lift coupler 150 is displaceable, relative to the room, via the displaceability of the patient lift coupler 150, relative to the mount 170, for exposing another portion of the room (e.g. a second portion of the room) to the UV radiation.

In some embodiments, for example, the displaceability of the patient lift coupler 150, relative to the mount 170, is effectuatable manually, for example, by applying a displacement force to the patient lift coupler 150.

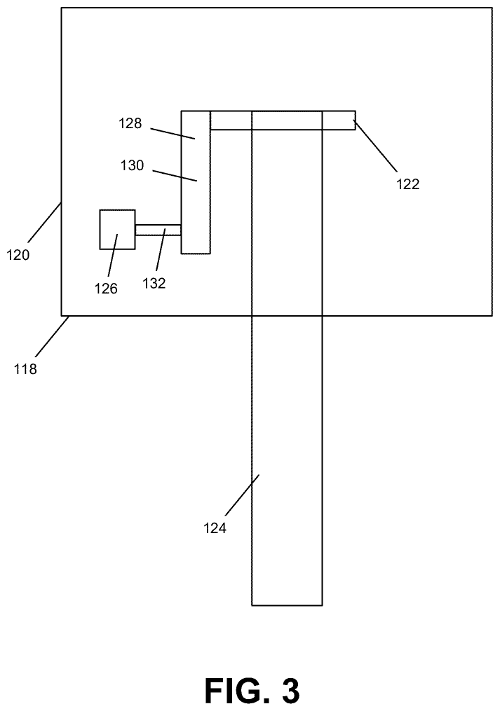

In some embodiments, for example, as depicted in FIG. 3, the patient lift 118 that is operably couplable to the patient lift coupler 150 includes a housing 120.

In some embodiments, for example, the patient lift 118 further includes a spool 122, which is connected to the housing 120. In some embodiments, for example, the spool 122 is connected to the housing 120 such that the spool 122 is rotatable, relative to the housing 120.

In some embodiments, for example, the patient lift 118 further includes a strap 124. The strap 124 is connected to the spool 122. In some embodiments, for example, the strap 124 is configured to connect to a patient supporter such as a sling or a seat, or configured to connect to a carry bar, which is configured to connect to the patient supporter.

In some embodiments, for example, the patient lift 118 further includes a drive unit 126. In some embodiments, for example, the drive unit 126 is operably coupled to the spool 122 such that the spool 122 is drivable by the drive unit 126 for winding the strap 124 onto the spool 122, with effect that the strap 124 is retracted, and further drivable by the drive unit 126 for unwinding the strap 124 from the spool 122, with effect that the strap 124 is extended. In some embodiments, for example, the drive unit 126 is a motor. In some embodiments, for example, the drive unit 126 is an electric motor. In some embodiments, for example, the drive unit 126 is a planetary motor. In some embodiments, for example, while the strap 124 is connected to a patient supporter, and the patient is supported on the patient supporter, the strap 124 is extendible, by unwinding the strap 124 from the spool 122, or retractable, by winding the strap 124 onto the spool 122, to displace the patient (e.g. to lift or lower the patient).

In some embodiments, for example, the patient lift 118 further includes a transmission configuration 128 to effectuate the operable coupling of the drive unit 126 and the spool 122, such that the winding of the strap 124 onto the spool 122 and the unwinding of the strap 124 from the spool 122 by the drive unit 126 is effectuatable by the transmission configuration 128. In this respect, the transmission configuration 128 is operably coupled to the drive unit 126, and further operably coupled to the spool 122. In some embodiments, for example, the transmission configuration 128 includes one or more transmission components 130, such as a gear, a wheel, and the like. In some embodiments, for example, one or more transmission components 130 is operably coupled to the spool 122 such that rotation of the one or more transmission components 130 effectuates rotation of the spool 122. In some embodiments, for example, the transmission configuration 128 includes a drive shaft 132 that is drivable by the drive unit 126, for effectuating rotation of the one or more transmission components 130. In some embodiments, for example, the transmission configuration 128 includes a chain, a belt, and the like, for operably coupling the one or more transmission components 130.

In some embodiments, for example, the patient lift 118 further includes an energy source. In some embodiments, for example, the energy source 112 is operably coupled to the drive unit 126 to provide energy to the drive unit 126.

In some embodiments, for example, the patient lift 118 further includes a controller for controlling the function or operation of the patient lift 118.

In some embodiments, for example, the displaceability of the patient lift coupler 150, relative to the mount 170, is such that the patient lift coupler 150 is displaceable, relative to the mount 170, along a displacement axis 166. In some embodiments, for example, the displacement axis 166 is a linear axis.

In some embodiments, for example, the mount 170 includes a guide 172, operably coupled to the patient lift coupler 150, and configured for guiding the displacement of the patient lift coupler 150 along the displacement axis 166. In some embodiments, for example, the mount 170 includes a track 173 that defines the guide 172, and the displacement axis 166 and a longitudinal axis 174 of the track 173 are disposed in a parallel relationship.

In some embodiments, for example, the guide 172 is a first guide 172, and the mount 170 further includes a second guide 176, wherein each one of the first guide 172 and the second guide 176, independently, are operably coupled to the patient lift coupler 150. In some embodiments, for example, the first guide 172 and the second guide 176 are co-operatively configured for guiding the displacement of the patient lift coupler 150 along the displacement axis 166. In some embodiments, for example, the mount 170 includes a first track 173 that defines the first guide 172 and a second track 177 that defines the second guide 176, and the displacement axis 166, a longitudinal axis 174 of the first track 173, and a longitudinal axis 178 of the second track 177, are disposed in a parallel relationship.

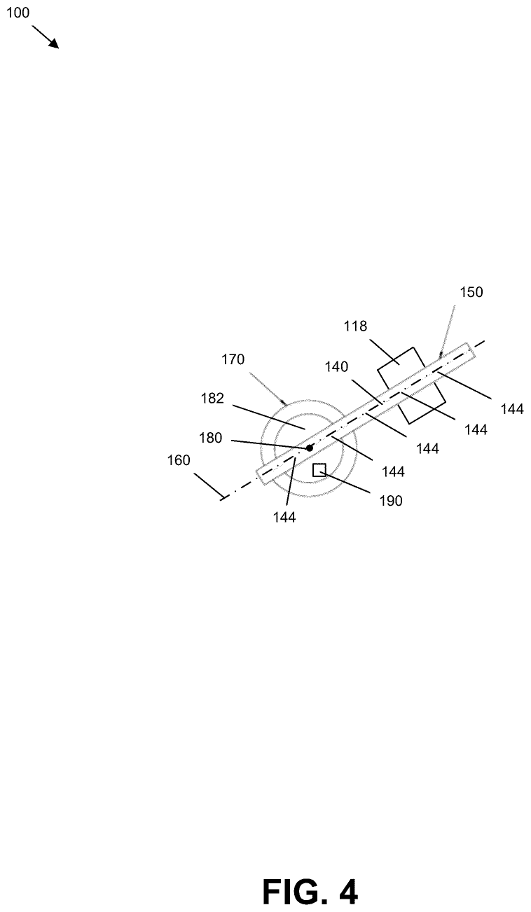

FIG. 4 depicts an alternate embodiment of the room irradiation configuration 100. As depicted, in some embodiments, for example, the displaceability of the patient lift coupler 150, relative to the mount 170, is such that the patient lift coupler 150 is rotatable, relative to the mount 170, about a rotation axis 180. In some embodiments, for example, the mount 170 includes a guide 182, operably coupled to the patient lift coupler 150, and configured for guiding the rotation of the patient lift coupler 150 about the rotation axis 180. In some embodiments, for example, the rotation axis 180, and a normal axis of a surface on which the mount 170 is mounted, are disposed in a parallel relationship.

In some embodiments, for example, the displaceability of the patient lift coupler 150, relative to the mount 170, is such that the patient lift coupler 150 is displaceable, relative to the mount 170, along a curvilinear path. In some embodiments, for example, the mount 170 includes a curved guide, operably coupled to the patient lift coupler 150, and configured for guiding the displacement of the patient lift coupler 150 along the curvilinear path.

In some embodiments, for example, the adjustable characteristic of the UV radiation emitted from the UV radiation configuration 140 includes a wavelength of the UV radiation. In some embodiments, for example, the adjusting of the wavelength of the UV radiation includes increasing the wavelength of the UV radiation. In some embodiments, for example, the adjusting of the wavelength of the UV radiation includes decreasing the wavelength of the UV radiation.

In some embodiments, for example, the UV radiation configuration 140 emits broad spectrum UV radiation. In some embodiments, for example, the UV radiation configuration 140 emits UV radiation of various wavelengths to have disinfection efficacy across a wide range of pathogens and microorganisms. In some embodiments, for example, the UV radiation configuration 140 emits Ultraviolet-C (UVC) radiation.

In some embodiments, for example, the adjustable characteristic of the UV radiation emitted from the UV radiation configuration 140 includes an intensity of the UV radiation. In some embodiments, for example, the adjusting of the intensity of the UV radiation includes increasing the intensity of the UV radiation. In some embodiments, for example, the adjusting of the intensity of the UV radiation includes decreasing the intensity of the UV radiation.

In some embodiments, for example, the room irradiation configuration 100 further comprises a drive unit 190. The mount 170, the drive unit 190, and the patient lift coupler 150 are co-operatively configured for effectuating the displacement of the patient lift coupler 150, relative to the mount 170. In some embodiments, for example, the controller 102 is operably coupled to the drive unit 190 to adjust the displacement of the patient lift coupler 150, relative to the mount 170. In some embodiments, for example, the drive unit 190 is a motor. In some embodiments, for example, the drive unit 190 is an electric motor. In some embodiments, for example, the drive unit 190 is a planetary motor.

In some embodiments, for example, the room irradiation configuration 100 further includes a transmission configuration to effectuate the displacement of the patient lift coupler 150, relative to the mount 170, via the drive unit 190. In this respect, the transmission configuration is operably coupled to the drive unit 190, and further operably coupled to the patient lift coupler 150. In some embodiments, for example, the transmission configuration includes one or more transmission components, such as a gear, a wheel, and the like. In some embodiments, for example, the transmission configuration includes a drive shaft that is drivable by the drive unit 190, for effectuating rotation of the one or more transmission components. In some embodiments, for example, the transmission configuration includes a chain, a belt, and the like, for operably coupling the one or more transmission components.

In some embodiments, for example, the adjusting of the displacement of the patient lift coupler 150, relative to the mount 170, is such that the displacement of the patient lift coupler 150, relative to the mount 170, is increased.

In some embodiments, for example, the adjusting of the displacement of the patient lift coupler 150, relative to the mount 170, is such that the displacement of the patient lift coupler 150, relative to the mount 170, is decreased.

In some embodiments, for example, the adjusting of the displacement of the patient lift coupler 150, relative to the mount 170, is such that there is an absence of displacement of the patient lift coupler 150, relative to the mount 170.

In some embodiments, for example, the adjusting of the displacement of the patient lift coupler 150, relative to the mount 170, is such that a direction of the displacement of the patient lift coupler 150, relative to the mount 170, is changed.

In some embodiments, for example, the room irradiation configuration 100 further includes the user interface 116. The user interface 116 is configured to receive an input representative of an environment of the room irradiation configuration 100, for example, from a user, and communicating the input to the controller 102.

In some embodiments, for example, the adjusting of the characteristic of the UV radiation emitted by the UV radiation configuration 140 is based at least in part on the environment of the room irradiation configuration 100. In some embodiments, for example, based on an input representative of an environment that has relatively high sanitation requirements (e.g. operating room), the intensity of the UV radiation emitted from the UV radiation configuration 140 is increased. In some embodiments, for example, based on an input representative of an environment that has relatively low sanitation requirements (e.g. patient room), the intensity of the UV radiation emitted from the UV radiation configuration 140 is decreased.

In some embodiments, for example, the adjusting of the displacement of the patient lift coupler 150, relative to the mount 170, is based at least in part on the environment of room irradiation configuration 100. In some embodiments, for example, based on an input representative of an environment that has relatively high sanitation requirements (e.g. operating room), the displacement of the patient lift coupler 150, relative to the mount 170, is decreased. In some embodiments, for example, based on an input representative of an environment that has relatively low sanitation requirements (e.g. patient room), the displacement of the patient lift coupler 150, relative to the mount 170, is increased.

In some embodiments, for example, the user interface 116 is configured to receive an input representative of an operating mode of the room irradiation configuration 100, and communicating the input to the controller 102. In some embodiments, for example, the adjusting of the characteristic of the UV radiation emitted by the UV radiation configuration 140 is based at least in part on the operating mode of the room irradiation configuration 100. In some embodiments, for example, the displacement of the patient lift coupler 150, relative to the mount 170, are based at least in part on the operating mode of the room irradiation configuration 100.

In some embodiments, for example, an operating mode of the room irradiation configuration 100 includes a continuous mode. In some embodiments, for example, the continuous mode can be selected via the user interface 116. While the room irradiation configuration 100 is operating in the continuous mode, the UV radiation configuration 140 emits UV radiation continuously, such that at least a portion of the room in which the room irradiation configuration 100 is disposed is exposed to the UV radiation continuously. In some embodiments, for example, while the room irradiation configuration 100 is operating in the continuous mode, the continuous emission of UV radiation on the portion of the room in which the room irradiation configuration 100 is disposed effectuates ongoing disinfection of the portion of the room in which the room irradiation configuration 100 is disposed. In some embodiments, for example, the operation of the room irradiation configuration 100 in the continuous mode is particularly useful in high-traffic environments where the room irradiation configuration 100 is frequently operated. In some embodiments, for example, while the room irradiation configuration 100 is in operation or is able to be operated, the room irradiation configuration 100 is disposed in the continuous mode. In some embodiments, for example, the continuous mode can be activated during periods when the room is not in use for a brief period of time. While the room irradiation configuration 100 is operating in the continuous mode, the UV radiation emitted by the UV radiation configuration 140 is adjusted to have increased intensity, and the displacement of the patient lift coupler 150, relative to the mount 170, is adjusted to have increased displacement.

In some embodiments, for example, an operating mode of the room irradiation configuration 100 includes a disinfection mode. In some embodiments, for example, the disinfection mode can be selected via the user interface 116. In some embodiments, for example, the disinfection mode can be activated during periods when the room is not in use for an extended period of time. While the room irradiation configuration 100 is operating in the disinfection mode, the UV radiation emitted by the UV radiation configuration 140 is adjusted to have increased intensity, and the displacement of the patient lift coupler 150, relative to the mount 170, is adjusted to have decreased displacement.

In some embodiments, for example, while the room is not in use or not operational, it is expected that a patient or a user is not nearby the UV radiation configuration 140, such that the risk of exposure of the patient or user to UV radiation emitted by the UV radiation configuration 140 is reduced. As such, in some embodiments, for example, while the room irradiation configuration 100 is disposed in the disinfection mode, the intensity of the UV radiation emitted by the UV radiation configuration 140 is increased.

In some embodiments, for example, the UV radiation configuration 140 comprises a UV radiation source 144 for emitting UV radiation.

In some embodiments, for example, adjusting the wavelength of the UV radiation emitted from the UV radiation configuration 140 includes adjusting the wavelength emitted from the UV radiation source 144. In some embodiments, for example, adjusting the intensity of the UV radiation emitted from the UV radiation configuration 140 includes adjusting the intensity emitted from the UV radiation source 144.

In some embodiments, for example, the UV radiation source 144 is a first UV radiation source 144, and the UV radiation configuration 140 further comprises a second UV radiation source 144 for emitting UV radiation.

In some embodiments, for example, adjusting the wavelength of the UV radiation emitted from the UV radiation configuration 140 includes adjusting the wavelength of the UV radiation emitted from one or more UV radiation sources 144. In some embodiments, for example, the wavelength of UV radiation emitted from a first UV radiation source 144 is the same as the wavelength of UV radiation emitted from a second UV radiation source 144. In some embodiments, for example, the wavelength of UV radiation emitted from the first UV radiation source 144 and wavelength of UV radiation emitted from the second UV radiation source 144 are different. In some embodiments, for example, adjusting the wavelength of the UV radiation emitted from the UV radiation configuration 140 includes adjusting the wavelength by turning on or off one or more UV radiation sources 144.

In some embodiments, for example, adjusting the intensity of the UV radiation emitted from the UV radiation configuration 140 includes adjusting the intensity of the UV radiation emitted from one or more UV radiation sources 144. In some embodiments, for example, the intensity of UV radiation emitted from a first UV radiation source 144 is the same as the intensity of UV radiation emitted from a second UV radiation source 144. In some embodiments, for example, the intensity of UV radiation emitted from the first UV radiation source 144 and intensity of UV radiation emitted from the second UV radiation source 144 are different. In some embodiments, for example, adjusting the intensity of the UV radiation emitted from the UV radiation configuration 140 includes adjusting the intensity by turning on or off one or more UV radiation sources 144.

In some embodiments, for example, the first UV radiation source 144 and the second UV radiation source 144 are disposed in a side-by-side relationship. In some embodiments, for example, the first UV radiation source 144 and the second UV radiation source 144 are disposed in opposing relationship.

In some embodiments, for example, the room irradiation configuration 100 includes a plurality of UV radiation sources 144, as depicted in FIG. 1. In some embodiments, for example, the wavelength and the intensity of the UV radiation emitted from the UV radiation configuration 140 are adjusted by turning on the desired UV radiation sources 144, such that the UV radiation emitted from the UV radiation sources 144 combine to generate the UV radiation of desired wavelength and intensity.

In some embodiments, for example, for each one of the UV radiation sources 144, independently, the UV radiation source is a UV light source. In some embodiments, for example, for each one of the UV radiation sources 144, independently, the UV radiation source is a light emitting diode (LED). In some embodiments, for example, the LED is a visible LED. In some embodiments, for example, the LED is a non-visible LED. In some embodiments, for example, for each one of the UV radiation sources 144, independently, the UV radiation source is a UV bulb. In some embodiments, for example, for each one of the UV radiation sources 144, independently, the UV radiation source 144 is a UV radiation emitter.

In some embodiments, for example, while the patient lift 118 is operably coupled to the patient lift coupler 150, the patient lift 118 is displaceable, relative to the patient lift coupler 150. In some embodiments, for example, while the patient lift 118 is operably coupled to the patient lift coupler 150, the patient lift 118 is displaceable, relative to the patient lift coupler 150, along a patient lift displacement axis 160. In some embodiments, for example, the displaceability of the patient lift 118, relative to the patient lift coupler 150, is such that the patient lift 118 is displaceable, relative to the mount 170, along the patient lift displacement axis 160. In some embodiments, for example, the patient lift displacement axis 160 is a linear axis.

In some embodiments, for example, the patient lift coupler 150 includes a guide 162, configured for operably coupling to the patient lift 118 and for guiding the displacement of the patient lift 118 along the patient lift displacement axis 160.

In some embodiments, for example, the patient lift coupler 150 includes a track 163 that defines the guide 162, and the patient lift displacement axis 160 and a longitudinal axis 164 of the track 163 are disposed in a parallel relationship.

In some embodiments, for example, the UV radiation configuration 140 is defined by a strip of UV radiation sources 144. In some embodiments, for example, the strip of UV radiation sources 144 extends along an axis that is parallel to the longitudinal axis 164 of the track 163.

In some embodiments, for example, the displaceability of the patient lift 118, relative to the patient lift coupler 150, is effectuatable manually, for example, by applying a displacement force to the patient lift 118.

In some embodiments, for example, the patient lift displacement configuration 110, for example, the mount 170, is configured to be mountable to a ceiling of the room. In some embodiments, for example, the patient lift displacement configuration 110, for example, the mount 170, is configured to be mountable to a wall of the room. In some embodiments, for example, the patient lift displacement configuration 110, for example, the mount 170, is configured to be mountable to a floor of the room. In some embodiments, for example, the mountability of the mount 170 to the room is such that, while the mount 170 is mounted to the room, the mount 170 is secured to the room, such that displacement of the mount 170, relative to the room, is opposed, for example, prevented, for example, absent.

In some embodiments, for example, the UV radiation configuration 140 is a first UV radiation configuration 1402, and the mount 170 comprises a second UV radiation configuration 1404 configured to emit UV radiation. In some embodiments, for example, the controller 102 is operably coupled to the second UV radiation configuration 1404 to adjust a characteristic of the UV radiation emitted by the second UV radiation configuration 1404. In some embodiments, for example, while the mount 170 is mounted to the room, and the second UV radiation configuration 1404 is emitting UV radiation: a portion of the room is exposed to the UV radiation emitted by the second UV radiation configuration 1404. In some embodiments, for example, the portion of the room is exposed to the UV radiation emitted by the first UV radiation configuration 1402 is different from the portion of the room is exposed to the UV radiation emitted by the second UV radiation configuration 1404. In this respect, in some embodiments, for example, the portion of the room is exposed to the UV radiation emitted by the first UV radiation configuration 1402 is a first portion of the room, and the portion of the room is exposed to the UV radiation emitted by the second UV radiation configuration 1404 is a second portion of the room.

In some embodiments, for example, as depicted in FIG. 1, the mount 170 includes a first guide 172 and a second guide 176. In some embodiments, for example, the first guide 172 includes the second UV radiation configuration 1404 and the second guide 176 includes a third UV radiation configuration 1406. In some embodiments, for example, the controller 102 is operably coupled to the third UV radiation configuration 1406 to adjust a characteristic of the UV radiation emitted by the third UV radiation configuration 1406. In some embodiments, for example, while the mount 170 is mounted to the room, and the third UV radiation configuration 1406 is emitting UV radiation: a portion of the room is exposed to the UV radiation emitted by the third UV radiation configuration 1406. In some embodiments, for example, the portion of the room is exposed to the UV radiation emitted by the first UV radiation configuration 1402 is different from the portion of the room is exposed to the UV radiation emitted by the third UV radiation configuration 1406. In some embodiments, for example, the portion of the room is exposed to the UV radiation emitted by the second UV radiation configuration 1404 is different from the portion of the room is exposed to the UV radiation emitted by the third UV radiation configuration 1406. In this respect, in some embodiments, for example, the portion of the room is exposed to the UV radiation emitted by the first UV radiation configuration 1402 is a first portion of the room, the portion of the room is exposed to the UV radiation emitted by the second UV radiation configuration 1404 is a second portion of the room, the portion of the room is exposed to the UV radiation emitted by the third UV radiation configuration 1406 is a third portion of the room.

In some embodiments, for example, each one of the first UV radiation configuration 1402, the second UV radiation configuration 1404, and the third UV radiation configuration 1406, independently, is defined by a strip of UV radiation sources 144. In some embodiments, for example, the strip of UV radiation sources 144 of the first UV radiation configuration 1402 extends along an axis that is parallel to the longitudinal axis 164 of the track 163. In some embodiments, for example, the strip of UV radiation sources 144 of the second UV radiation configuration 1404 extends along an axis that is parallel to the longitudinal axis 174 of the track 173. In some embodiments, for example, the strip of UV radiation sources 144 of the third UV radiation configuration 1406 extends along an axis that is parallel to the longitudinal axis 178 of the track 177.

In some embodiments, for example, the axis 164 and the axis 174 are disposed in a non-parallel relationship, such as a perpendicular relationship, as depicted in FIG. 1. In some embodiments, for example, the axis 164 and the axis 178 are disposed in a non-parallel relationship, such as a perpendicular relationship, as depicted in FIG. 1. In some embodiments, for example, the axis 174 and the axis 178 are disposed in a parallel relationship, as depicted in FIG. 1.

In some embodiments, for example, while the room irradiation configuration 100 is mounted to the room, for example, via mounting of the mount 170 to the room (e.g. while the mount 170 is mounted to the room), the UV radiation configuration 140 is displaceable, relative to the room, via the displaceability of the patient lift coupler 150, relative to the mount 170, for exposing one or more portions of the room to the UV radiation. In some embodiments, for example, wherein the room irradiation configuration 100 includes more than one UV radiation configuration, for example, wherein the room irradiation configuration 100 includes the first UV radiation configuration 1402, the second UV radiation configuration 1404, and the third UV radiation configuration 1406, as depicted in FIG. 1, while the room irradiation configuration 100 is mounted to the room, at least one of the UV radiation configurations is displaceable, relative to the room, via the displaceability of the patient lift coupler 150, relative to the mount 170 (in the room irradiation configuration 100 as depicted in FIG. 1, the first UV radiation configuration 1402 is displaceable, relative to the room, via the displaceability of the patient lift coupler 150, relative to the mount 170), for exposing one or more portions of the room to the UV radiation.

In some embodiments, for example, wherein the room irradiation configuration 100 includes more than one UV radiation configuration, for example, wherein the room irradiation configuration 100 includes the first UV radiation configuration 1402, the second UV radiation configuration 1404, and the third UV radiation configuration 1406, as depicted in FIG. 1, while the room irradiation configuration 100 is mounted to the room, for example, via mounting of the mount 170 to the room (e.g. while the mount 170 is mounted to the room), at least one of the UV radiation configurations is displaceable, relative to the room, via the displaceability of the patient lift coupler 150, relative to the mount 170 (in the room irradiation configuration 100 as depicted in FIG. 1, the first UV radiation configuration 1402 is displaceable, relative to the room, via the displaceability of the patient lift coupler 150, relative to the mount 170), for exposing one or more portions of the room to the UV radiation, and there is an opposition, for example, prevention, for example, absence, of displacement of at least one of the UV radiation configurations, relative to the room, via the mounting of the mount 170 to the room.

In some embodiments, for example, wherein the room irradiation configuration 100 includes more than one UV radiation configuration, while the room irradiation configuration 100 is mounted to the room, for example, via mounting of the mount 170 to the room (e.g. while the mount 170 is mounted to the room), a portion of the room can be exposed to UV radiation by two or more UV radiation configurations. In some embodiments, for example, as depicted in FIG. 1, a portion of the room can be exposed to UV radiation by the first UV radiation configuration 1402 and by the second UV radiation configuration 1404. In some embodiments, for example, as depicted in FIG. 1, a portion of the room can be exposed to UV radiation by the first UV radiation configuration 1402 and by the third UV radiation configuration 1406. In some embodiments, for example, a portion of the room can be exposed to UV radiation by the second UV radiation configuration 1404 and by the third UV radiation configuration 1406. In some embodiments, for example, a portion of the room can be exposed to UV radiation by the first UV radiation configuration 1402, by the second UV radiation configuration 1404, and by the third UV radiation configuration 1406.

In some embodiments, for example, the room irradiation configuration 100 includes safety features for reducing the risk of accidental exposure of the UV radiation to users. In some embodiments, for example, the room irradiation configuration 100 includes an emergency pull cord. In some embodiments, for example, in response to pulling on the emergency pull cord, the controller 102 sends a control command to the UV radiation configuration 140 to stop emission of UV radiation. In some embodiments, for example, in response to pulling on the emergency pull cord, the controller 102 sends a control command to the drive unit 126 to stop driving of the spool 122. In some embodiments, for example, in response to pulling on the emergency pull cord, the controller 102 sends a control command to the drive unit 190 to stop displacement of the patient lift coupler 150, relative to the mount 170.

In some embodiments, for example, a user can control the room irradiation configuration 100 via a hand control unit or a control panel of the user interface 116, which is disposed in operable configuration with the controller 102. In some embodiments, for example, the hand control unit is a hand control unit of the room irradiation configuration 100. In some embodiments, for example, the hand control unit is a hand control unit of the patient lift 118 that is operably coupled to the patient lift coupler 150. In some embodiments, for example, the control panel is a control panel of the room irradiation configuration 100 that is mounted to a wall of the room in which the room irradiation configuration 100 is disposed. The user can select the mode that the room irradiation configuration 100 is to operate in, and initiate the operation of the room irradiation configuration 100. In response to the selection of the operating mode, the wavelength and intensity of the UV radiation emitted by the one or more UV radiation configurations, for example, UV radiation configurations 1402, and 1404, and 1406, are adjusted, and the displacement of the patient lift coupler 150, relative to the mount 170, is adjusted, for exposing the room to UV radiation emitted by the one or more UV radiation configurations having the desired characteristics for the desired duration. The user can further pause or stop the operation of the room irradiation configuration 100 via the hand control unit or the control panel. In some embodiments, for example, after the operation of the room irradiation configuration 100 is initiated, a pre-determined amount of time is allowed to pass, before the one or more UV radiation configurations begins to emit UV radiation, which allows time for an operator to leave the room in which the room irradiation configuration 100 is disposed. In some embodiments, for example, the status of the room irradiation configuration 100 is displayed on the user interface 116, for example, on the hand control unit or the control panel. In some embodiments, for example, the status of the operation of the room irradiation configuration 100 is displayed on the user interface 116, for example, on the hand control unit or the control panel. In some embodiments, for example, an alert, for example, an audible or visible alert, is provided by the user interface 116, to indicate to a user that the one or more UV radiation configurations are, or will be, emitting UV radiation. In some embodiments, for example, while the one or more UV radiation configurations is emitting UV radiation, the controller 102 will send a control command to the one or more UV radiation configurations to stop emission of the UV radiation after a pre-determined amount of time has passed. In such embodiments, for example, the UV radiation is emitted for the predetermined amount of time to achieve the desired exposure of the room to the UV radiation, and the UV radiation is not emitted more than desired or necessary.

In some embodiments, for example, the room irradiation configuration 100 includes visible warnings, such as labels and signs, to indicate to a user or bystander that the room irradiation configuration 100 is able to emit UV radiation, and to identify potential hazards of UV radiation and appropriate precautions that can be taken.

In the preceding description, for purposes of explanation, numerous details are set forth in order to provide a thorough understanding of the embodiments; however the specific details are not necessarily required. In other instances, well-known electrical structures and circuits are shown in block diagram form in order not to obscure the understanding. For example, specific details are not provided as to whether the embodiments described herein are implemented as a software routine, hardware circuit, firmware, or a combination thereof.

The steps and/or operations in the flowcharts and drawings described herein are for purposes of example only. There may be many variations to these steps and/or operations without departing from the teachings of the present disclosure. For instance, the steps may be performed in a differing order, or steps may be added, deleted, or modified.

The coding of software for carrying out the above-described methods described for execution by a controller (or processor) or other apparatus is within the scope of a person of ordinary skill in the art having regard to the present disclosure. Machine readable code executable by one or more processors of one or more respective devices to perform the above-described method may be stored in a machine readable medium such as the memory of the data manager. The terms “software” and “firmware” are interchangeable within the present disclosure and comprise any computer program stored in memory for execution by a processor, comprising RAM memory, ROM memory, erasable programmable ROM (EPROM) memory, electrically EPROM (EEPROM) memory, and non-volatile RAM (NVRAM) memory. The above memory types are example only, and are thus not limiting as to the types of memory usable for storage of a computer program.

All values and sub-ranges within disclosed ranges are also disclosed. In addition, although the systems, devices and processes disclosed and shown herein may comprise a specific plurality of elements/components, the systems, devices, configurations, and assemblies may be modified to comprise additional or fewer of such elements/components. For example, although any of the elements/components disclosed may be referenced as being singular, the embodiments disclosed herein may be modified to comprise a plurality of such elements/components. The subject matter described herein intends to cover and embrace all suitable changes in technology.

Although the present disclosure is described, at least in part, in terms of methods, a person of ordinary skill in the art will understand that the present disclosure is also directed to the various components for performing at least some of the aspects and features of the described methods, be it by way of hardware (DSPs, ASIC, or FPGAs), software or a combination thereof. Accordingly, the technical solution of the present disclosure may be embodied in a non-volatile or non-transitory machine readable medium (e.g., optical disk, flash memory, etc.) having stored thereon executable instructions tangibly stored thereon that enable a processing device (e.g., a data manager) to execute examples of the methods disclosed herein.

The term “processor” may comprise any programmable system comprising systems using micro-or nano-processors/controllers, reduced instruction set circuits (RISC), application specific integrated circuits (ASICs), logic circuits, and any other circuit or processor capable of executing the functions described herein. The term “database” may refer to either a body of data, a relational database management system (RDBMS), or to both. As used herein, a database may comprise any collection of data comprising hierarchical databases, relational databases, flat file databases, object-relational databases, object oriented databases, and any other structured collection of records or data that is stored in a computer system. The above examples are example only, and thus are not intended to limit in any way the definition and/or meaning of the terms “processor” or “database”.

The present disclosure may be embodied in other specific forms without departing from the subject matter of the claims. The described example embodiments are to be considered in all respects as being only illustrative and not restrictive. The present disclosure intends to cover and embrace all suitable changes in technology. The scope of the present disclosure is, therefore, described by the appended claims rather than by the foregoing description. The scope of the claims should not be limited by the embodiments set forth in the examples, but should be given the broadest interpretation consistent with the description as a whole.

1.-34. (canceled)

35. A room irradiation configuration configured for exposing a room to ultraviolet (UV) radiation, comprising:
a patient lift displacement configuration, comprising:
a mount configured to be mounted to the room;
a patient lift coupler, operably coupled to the mount such that the patient lift coupler is displaceable, relative to the mount, and is configured to operably couple to a patient lift such that the patient lift is displaceable, relative to the patient lift coupler, the patient lift coupler comprising a UV radiation configuration configured to emit UV radiation;
a controller operably coupled to the UV radiation configuration to adjust a characteristic of the UV radiation emitted by the UV radiation configuration;
wherein, while the mount is mounted to the room, and the UV radiation configuration is emitting UV radiation:
a first portion of the room is exposed to the UV radiation; and
the patient lift coupler is displaceable, relative to the room, via the displaceability of the patient lift coupler, relative to the mount, for exposing another portion of the room to the UV radiation.

36. The room irradiation configuration of claim 35, wherein the displaceability of the patient lift coupler, relative to the mount, is such that the patient lift coupler is displaceable, relative to the mount, along a displacement axis.

37. The room irradiation configuration of claim 36, wherein the displacement axis is a linear axis.

38. The room irradiation configuration of claim 36, wherein the mount includes a guide, operably coupled to the patient lift coupler, and configured for guiding the displacement of the patient lift coupler along the displacement axis.

39. The room irradiation configuration of claim 38, wherein the mount includes a track that defines the guide, and the displacement axis and a longitudinal axis of the track are disposed in a parallel relationship.

40. The room irradiation configuration of claim 36, wherein the mount includes a first guide and a second guide, wherein each one of the first guide and the second guide, independently, are operably coupled to the patient lift coupler, and co-operatively configured for guiding the displacement of the patient lift coupler along the displacement axis.

41. The room irradiation configuration of claim 40, wherein the mount includes a first track that defines the first guide and a second track that defines the second guide, and the displacement axis, a longitudinal axis of the first track, and a longitudinal axis of the second track, are disposed in a parallel relationship.

42. The room irradiation configuration of claim 35, wherein the displaceability of the patient lift coupler, relative to the mount, is such that the patient lift coupler is rotatable, relative to the mount, about a rotation axis.

43. The room irradiation configuration of claim 42, wherein the mount includes a guide, operably coupled to the patient lift coupler, and configured for guiding the rotation of the patient lift coupler about the rotation axis.

44. The room irradiation configuration of claim 35, wherein the displaceability of the patient lift coupler, relative to the mount, is such that the patient lift coupler is displaceable, relative to the mount, along a curvilinear path.

45. The room irradiation configuration of claim 44, wherein the mount includes a curved guide, operably coupled to the patient lift coupler, and configured for guiding the displacement of the patient lift coupler along the curvilinear path.

46. The room irradiation configuration of claim 35, wherein the adjustable characteristic of the UV radiation includes a wavelength of the UV radiation.

47. The room irradiation configuration of claim 35, wherein the adjustable characteristic of the UV radiation includes an intensity of the UV radiation.

48. The room irradiation configuration of claim 35, further comprising:
a drive unit, wherein the mount, the drive unit, and the patient lift coupler are co-operatively configured for effectuating the displacement of the patient lift coupler, relative to the mount;
wherein the controller is operably coupled to the drive unit to adjust the displacement of the patient lift coupler, relative to the mount.

49. The room irradiation configuration of claim 48, further comprising:
a user interface, configured to receive an input representative of an environment of the room irradiation configuration, wherein the adjusting of the characteristic of the UV radiation is based at least in part on the environment of the room irradiation configuration, and the adjusting of the displacement of the patient lift coupler, relative to the mount, are based at least in part on the environment of the room irradiation configuration.

50. The room irradiation configuration of claim 48, further comprising:
a user interface, configured to receive an input representative of an operating mode of the room irradiation configuration, wherein the adjusting of the characteristic of the UV radiation is based at least in part on the operating mode of the room irradiation configuration, and the adjusting of the displacement of the patient lift coupler, relative to the mount, is based at least in part on the operating mode of the room irradiation configuration.

51. The room irradiation configuration of claim 35, wherein the displaceability of the patient lift, relative to the patient lift coupler, is such that the patient lift is displaceable, relative to the patient lift coupler, along a patient lift displacement axis.

52. The room irradiation configuration of claim 35, wherein the mount is configured to be mountable to a ceiling of the room.

53. The room irradiation configuration of claim 35, wherein:
the UV radiation configuration is a first UV radiation configuration;
the mount comprises a second UV radiation configuration configured to emit UV radiation; and
the controller is operably coupled to the second UV radiation configuration to adjust a characteristic of the UV radiation emitted by the second UV radiation configuration;
wherein, while the mount is mounted to the room, and the second UV radiation configuration is emitting UV radiation:
a second portion of the room is exposed to the UV radiation emitted by the second UV radiation configuration.

54. The room irradiation configuration of claim 53, wherein:
the mount comprises a third UV radiation configuration configured to emit UV radiation; and
the controller is operably coupled to the third UV radiation configuration to adjust a characteristic of the UV radiation emitted by the third UV radiation configuration;
wherein, while the mount is mounted to the room, and the third UV radiation configuration is emitting UV radiation:
a third portion of the room is exposed to the UV radiation emitted by the third UV radiation configuration.
